(19) United States
(12) Patent Application Publication
(10) Pub. No.: US 2005/0031608 A1
(43) Pub. Date: Feb. 10, 2005

(54) Modified alpha-neurotoxins as painkillers

(76) Inventors: Paul Reid, Ft. Lauderdale, FL (US); Laurence Raymond, Ft. Lauderdale, FL (US)

Correspondence Address: Robert J. Van Der Wall;Colonial Bank Centre, Suite 1620, 1200 Brickell Avenue, Miami, FL, 33131, US

(21) Appl. No.: 10/636,458

(22) Filed: Aug. 6, 2003

(51) Int. Cl. A61K 38/48; A61K 38/46

(52) U.S. Cl. 424/94.65

(57) ABSTRACT

The disclosed invention is a composition of matter, a process of production thereof, and a method for the treatment of chronic pain, especially to the treatment of heretofore intractable pain as associated with advanced cancer, neurological conditions and rheumatoid arthritis. The treatment of pain associated with viral infections and lesions are also within the contemplation of the present invention. The composition of matter comprises modified alpha-neurotoxins or modified venoms known to contain alpha-neurotoxins in an acceptable carrier for either parenteral, oral or topical administration.

### BACKGROUND OF THE INVENTION

1. Field of the Invention

The present invention relates to a class of proteins, a process of production thereof, and a method for the treatment of chronic pain, especially to the treatment of heretofore intractable pain as associated with advanced cancer, neurological conditions and rheumatoid arthritis. The pain associated with viral infections and lesions may also respond to treatment with the present invention. The composition consists of modified alpha-neurotoxins or modified venoms known to contain alpha-neurotoxins in an acceptable carrier for either parenteral, oral or topical administration.

2. Description of the Prior Art

Sanders et al. had commenced investigating the application of modified venoms to the treatment of ALS in 1953 having employed poliomyelitis infection in monkeys as a model. Other antiviral studies had reported inhibition of pseudo rabies (a herpes virus) and Semliki Forest virus (alpha-virus). See Sanders' U.S. Pat. Nos. 3,888,977, 4,126,676, and 4,162,303. Sanders justified the pursuit of this line of research through reference to the studies of Lamb and Hunter (1904) though it is believed that the original idea was postulated by Haast. See Haast U.S. Pat. Nos. 4,741,902 and 5,723,477. The studies of Lamb and Hunter (Lancet 1:20, 1904) showed by histopathologic experiments with primates killed by neurotoxic Indian cobra venom that essentially all of the motor nerve cells in the central nervous system were involved by this venom. A basis of Sanders' invention was the discovery that such neurotropic snake venom, in an essentially non-toxic state, also could reach that same broad spectrum of motor nerve cells and block or interfere with invading pathogenic bacteria, viruses or proteins with potentially deleterious functions. Thus, the snake venom used in producing the composition was a neurotoxic venom, i.e. causing death through neuromuscular blockade. As the dosages of venom required to block the nerve cell receptors would have been far more than sufficient to quickly kill the patient, it was imperative that the venom was detoxified. The detoxified but undenatured venom was referred to as being neurotropic. The venom was preferably detoxified in the mildest and most gentle manner. While various detoxification procedures were known then to the art, such as treatment with formaldehyde, fluorescein dyes, ultraviolet light, ozone, heat, it was preferred that gentle oxygenation at relatively low temperatures be practiced, although the particular detoxification procedure was not defined as critical. Sanders employed a modified Bouquet detoxification procedure using hydrogen peroxide, outlined below. The acceptability of any particular detoxification procedure was tested by the classical Semliki Forest virus test, as taught by Sanders, U.S. Pat. No. 4,162,303.

The first unpublished, clinical study approved in 1983 by the Human Subjects Committee of the University of Miami was a small Phase I study in 5 patients with genital herpes infections (BB-IND1670). The purpose was to convince clinical investigators there of the claimed safety of the modified venom material. All patients tolerated the intramuscular dosages well and there was no evidence of allergic or other local or systemic reactions. The patients reported that the pain and itching regressed faster than previously experienced and this was reflected objectively by the rates of reduction in virus titers among lesions scrapings in the two patients whose lesions contained measurable numbers of infectious particles. In 1995, a study by Vargas and Cortes was published in which 78 patients with various herpetic infections were treated with the injection of modified cobratoxin, the object of the study being an investigation into the antiviral properties of modified cobratoxin. A topical formulation of the drug was also employed, consisting of 0.35 mg of the modified cobratoxin per 100 g of cream base, with moderate success.

In August of 1984 an application for an Intrastate Investigational Drug (FSDHRS Protocol RA-1(002) from the Department of Health and Rehabilitation (HRS) in Florida was approved which permitted the study of oxidized venoms in patients with Rheumatoid Arthritis (RA). A total of 13 patients, ranging in age from 49 to 81, were enrolled for a period of 4 weeks. The protocol's criteria for patient entry was; 1.) Active “classical” RA as defined by the Am. Rheu. Assn.; 2.) Patients refractory to conventional RA second stage drugs; 3.) The investigators must have seen the patient in their practice of rheumatology. The formulation was administered IM daily and was well tolerated. Improvement (30-49%) was seen in the range of joint motion, early morning stiffness and stamina. Only injection site reactions in some patients were noted and were controlled with anti-histamines. A subjective reduction in pain associated with RA as a clinical endpoint was not included in the trial protocol and no reference was made to any amelioration of pain.

The production of drug product by Dr. M. Sanders was achieved using hydrogen peroxide as the oxidizing agent in addition to other components utilizing the recipe he employed for over 30 years (Sanders et al., 1975, 1978). This method was patented (U.S. Pat. Nos. 3,888,977 and 4,126,676) and published by Sanders on several occasions. The last patent expired in 1994. Furthermore, several techniques have been developed for modifying neurotoxins to yield a potentially therapeutic product though many have not been reduced to practice. These have included hydrogen peroxide, ozone, performic acid, iodoacetamide and iodoacetic acid. Some of these procedures have been published and others patented. Obviously some procedures are easier than others to utilize and the focus for commercial production has been on the simpler methods.

U.S. Pat. No. 5,364,842 describes the use of omega-conopeptides having defined binding/inhibitory properties in the treatment of chronic pain. In that patent is described omega-conopeptides having related inhibitory and binding activities that enhance the effects of opioid compounds in producing analgesia in mammalian subjects. In addition, these compounds may also produce analgesia in the absence of opioid treatment. Another requisite property of anti-nociceptive omega-conotoxin compounds, in accordance with the invention, is their ability to specifically inhibit depolarization-evoked and calcium-dependent neuro-transmitter release from neurons. In the case of anti-nociceptive omega-conopeptides, inhibition of electrically stimulated release of acetylcholine at the myenteric plexus of the guinea pig ileum is predictive of anti-nociceptive activity. U.S. Pat. No. 6,399,574 similarly describes the use of conantokin peptides which are antagonists of the NMDA receptor. However, these peptides must be delivered intrathecally in order to be effective.

Other references of interest include two patents, Haast, U.S. Pat. No. 4,341,762; Cosford, et al., U.S. Pat. No. 5,585,388, which claims compounds as modulators of acetylcholine receptors. Literature references of interest are: Chuang L. Y., Lin S. R., Chang S. F. and Chang C. C. Toxicon 27:211-219 (1989); Dierks R. E., Murphy F. A., and Harrison A. K. Am. J. Pathol. 54: 251-274 (1969); Hudson R A, Montgomery I N and Rauch H C. Mol Immunol. (1983) February; 20(2):229-32; Lamb, G and Hunter, W. K, The Lancet, 1: 20-22; Marx, A., Kirckner, T., Hoppe, F., O'Connor, R., Schalke, B., Tzartos, S. and Muller-Hermelink, H. K., Amer. J. Path, (1989) 134, No. 4, 865-75; Miller, K., Miller, G. G., Sanders, M. And Fellowes, O. N., Biophys et Biophysica Acta 496:192-196) (1977); Sanders, M., Soret, M. G. and Akin, B. A.; Ann. N.Y. Acad. Sci. 53: 1-12 (1953); Sanders, M., Soret, G., and Akin, B. A.; J. Path. Bacteriol. 68:267-271 (1954); Sanders M. And Fellows O.; Cancer Cytology 15:34-40(1975) and in Excerpta Medica International; Congress Series No. 334 containing abstracts of papers presented at the III International Congress of Muscle Diseases, Newcastle on Tyne, September 1974; Sanders M., Fellowes O. N. and Lenox A. C.; In: Toxins: Animal, Plant and Microbial, Proceedings of the fifth international symposium; P. Rosenberg, editor, Pergamon Press, New York 1978, p. 481; Tseng, L. F., Chiu, T. H., and Lee, C. Y.; Tox. Appl. Pharmac. 12:526-535 (1968); Tsiang H., de la Porte S., Ambroise D. J., Derer M. And Koenig J.; J. Neuropathol. Exp. Neurol. 45: 28-42; Tu A. T.; Ann. Rev. Biochem. 42:235-258(1973); Carstens E, Anderson K A, Simons C T, Carstens M I, Jinks S L. Psychopharmacology (Berl) 2001 August;157(1):40-5 “Analgesia induced by chronic nicotine infusion in rats: differences by gender and pain test.”; Damaj, M. I., Fei-Yin, M., Dukat, M., Glassco, W., Glennon, R. A. and Martin, B. R., JPET 1998 284:1058-1065, “Antinociceptive Responses to Nicotinic Acetylcholine Receptor Ligands after Systemic and Intrathecal Administration in Mice.”; Damaj M I, Meyer E M, Martin B R. Neuropharmacology 2000 October;39(13):2785-91 “The antinociceptive effects of alpha7 nicotinic agonists in an acute pain model.”; Decker M W, Meyer M D, Sullivan J P. Expert Opin Investig Drugs 2001 October;10(10):1819-30 “The therapeutic potential of nicotinic acetylcholine receptor agonists for pain control.”; *Irnaten M, Wang J, Venkatesan P, Evans C, K Chang K S, Andresen M C, Mendelowitz D. Anesthesiology 2002 March;96(3):667-74 “Ketamine inhibits presynaptic and postsynaptic nicotinic excitation of identified cardiac parasympathetic neurons in nucleus ambiguus.”; Lieb K, Treffurth Y, Berger M, Fiebich B L. Neuropsychobiology 2002;45 Suppl 1:2-6 “Substance P and affective disorders: new treatment opportunities by neurokinin 1 receptor antagonists?”; Min C K, Owens J, Weiland G A. Mol Pharmacol 1994 February;45(2):221-7 “Characterization of the binding of [3H]substance P to the nicotinic acetylcholine receptor of Torpedo electroplaque.”; Schaible H G, Ebersberger A, Von Banchet G S. Ann N Y Acad Sci 2002 June;966:343-354 “Mechanisms of Pain in Arthritis.” ; Schmidt B L, Tambeli C H, Gear R W, Levine J D. Neuroscience 2001;106(1):129-36 “Nicotine withdrawal hyperalgesia and opioid-mediated analgesia depend on nicotine receptors in nucleus accumbens.”; *Shiraishi M, Minami K, Uezono Y, Yanagihara N, Shigematsu A, Shibuya I. Br J Pharmacol 2002 May;136(2):207-16 “Inhibitory effects of tramadol on nicotinic acetylcholine receptors in adrenal chromaffin cells and in Xenopus oocytes expressing alpha7 receptors.”

### SUMMARY OF THE INVENTION

It is an object of the invention to provide a composition and method for treating pain associated with advanced cancer, neuropathy, painful viral infections and their lesions in addition to rheumatic pain.

It is a further object of the invention to provide a composition and therapy for the treatment of pain of the aforementioned type, whose composition and therapy are safe, effective and may be administered over long periods of time.

Another object of the invention is to provide a method of manufacture of the composition of the present invention.

Other objects will be apparent to those skilled in the art from the following disclosures and claims to be later appended.

The present invention accomplishes the above-stated objectives, as well as others, as may be determined by a fair reading and interpretation of the entire specification. The modified venoms may be derived from various species including certain genera of snakes and Conus snails and are prepared by detoxification of the whole venom, neurotoxic fractions or specific neurotoxins contained in whole venom.

### DETAILED DESCRIPTION OF THE PREFERRED EMBODIMENTS

As required, detailed embodiments of the present invention are disclosed herein; however, it is to be understood that the disclosed embodiments are merely exemplary of the invention which may be embodied in various forms. Therefore, specific functional details disclosed herein are not to be interpreted as limiting, but merely as a basis for the claims to be later appended and as a representative basis for teaching one skilled in the art to variously employ the present invention in virtually any appropriate circumstance.

Chronic or intractable pain, such as may occur in conditions such as degenerative bone diseases and cancer, is a debilitating condition which is treated with a variety of analgesic agents, and often opioid compounds, such as morphine.

In general, brain pathways governing the perception of pain are still incompletely understood. Sensory afferent synaptic connections to the spinal cord, termed “nociceptive pathways” have been documented in some detail. In the first leg of such pathways, C- and A-fibers which project from peripheral sites to the spinal cord carry nociceptive signals. Polysynaptic junctions in the dorsal horn of the spinal cord are involved in the relay and modulation of sensations of pain to various regions of the brain, including the periaqueductal grey region (McGeer). Analgesia, or the reduction of pain perception, can be effected directly by decreasing transmission along such nociceptive pathways. Analgesic opiates are thought to act by mimicking the effects of endorphin or enkephalin peptide-containing neurons, which synapse pre-synaptically at the C- or A-fiber terminal and which, when they fire, inhibit release of glutamate and substance P. The key transmitter is glutamate that activates N-methyl-d-aspartate (NMDA) and non-NMDA receptors on spinal cord neurons. Substance P(SP) is a neuropeptide which is abundant in the periphery and the central nervous system, where it is colocalized with other neurotransmitters such as serotonin or dopamine. SP has been proposed to play a role in the regulation of pain including migraine and fibromyalgia, asthma, inflammatory bowel disease, emesis, psoriasis as well as in central nervous system disorders.

The synthesis of analgesics, particularly of morphine-like compounds, has always been a point of major interest in drug research. For decades, scientists throughout the world have attempted to develop effective analgesics by “re-building” the morphine molecule, considering its constitution a combination of certain “basic skeletons” from which they started their syntheses. Meperidine hydrochloride (also known as Dolantin or Demerol) is one such synthetic narcotic analgesic. It is one-tenth as potent an analgesic as morphine and its analgesic effect is halved again when given orally rather than parenterally. The onset of activity occurs within 10-45 minutes with a duration of 2-4 hours. It has superceded morphine as the preferred analgesic for moderate to severe pain. It has been found to be particularly useful for minor surgery, as in orthopedics, ophthalmology, rhinology, laryngology, and dentistry. It is also used in parenteral form for preoperative medication, adjunct to anesthesia and obstetrical analgesia. Like morphine, its binding to opioid receptors produces both psychologic and physical dependence with overdosing causing severe respiratory depression in addition to a number of other undesirable side effects and drug interactions.

Although calcium blocking agents, including a number of L-type calcium channel antagonists, have been tested as adjunct therapy to morphine analgesia, positive results are attributed to direct effects on calcium availability, since calcium itself is known to attenuate the analgesic effects of certain opioid compounds (Ben-Sreti). EGTA, a calcium chelating agent, is effective in increasing the analgesic effects of opioids. Moreover, in some cases, results from tests of calcium antagonists as adjunct therapy to opioids have been contradictory; some L-type calcium channel antagonists have been shown to increase the effects of opioids, while others of these compounds have been shown to decrease opioid effects (Contreras).

Due to the limitations of such analgesics, a number of novel alternatives are currently under investigation, including neuronal nicotinic acetylcholine receptor (nAChR) agonists. Acute administration of nicotine induces analgesia with subsequent development of tolerance. Interestingly, in nicotine-naive rats, injection of the nicotinic receptor antagonist mecamylamine into the nucleus accumbens (where the site for activity of substances of abuse such as opioids has been implicated in pain modulation) blocked antinociception produced by either systemic morphine, intra-accumbens co-administration of a mu- and a delta-opioid receptor agonist, or noxious stimulation (i.e., subdermal capsaicin in the hindpaw). Intra-accumbens mecamylamine by itself precipitated significant hyperalgesia in nicotine-tolerant rats which could be suppressed by noxious stimulation as well as by morphine. Maneckjee et al found that in-vitro that lung cancer cell growth could be suppressed by opioids and this activity was antagonized by nicotine. Thus, nicotinic receptors have been found to play a role in modulating pain transmission in the CNS. Activation of other cholinergic pathways by nicotine and nicotinic agonists has been shown to elicit antinociceptive effects in a variety of species and pain tests. During the 1990s, the discovery of the antinociceptive properties of the potent nAChR agonist epibatidine in rodents sparked interest in the analgesic potential of this class of compounds (Decker et al., 2001). The identification of considerable nAChR diversity suggested that the toxicities and therapeutic actions of the compound might be mediated by distinct receptor subtypes and, accordingly, epibatidine and its derivatives identified nicotinic acetylcholine receptors with mainly alpha4 receptors though receptors with alpha3 were also sensitive to these compounds. The involvement of alpha7 nicotinic receptors in nicotinic analgesia has been assessed through spinal (i.t.) and intraventricular (i.c.v.) administration in mice. Dose-dependent antinociceptive effects were seen with the alpha7 agonist choline after spinal and supraspinal injection using the tail-flick test (Damaj et al., 2000). Furthermore, alpha7 antagonists MLA and alpha-bungarotoxin significantly blocked the effects of choline. These studies suggested that activation of alpha7 receptors in the CNS elicits antinociceptive effects in an acute thermal pain model.

However, in contradiction of the above, nicotinic antagonists may also have a role in pain relief. Tramadol and Ketamine have been used clinically as analgesics however, until recently, their mechanism of analgesic effect was unknown. Studies showed that Tramadol inhibited nicotinic currents carried by alpha7 receptors expressed in Xenopus oocytes (Shiraishi et al.). It also inhibited both alpha-bungarotoxin-sensitive and -insensitive nicotinic currents in bovine adrenal chromaffin cells. It was concluded that tramadol inhibited catecholamine secretion partly by inhibiting nicotinic AChR functions. The alpha7 subtype was one of those inhibited by tramadol. Ketamine was found to inhibit the nicotine-evoked presynaptic facilitation of glutamate release (Irnaten et al.). Alpha-bungarotoxin, an antagonist of alpha7 containing nicotinic presynaptic receptors, blocked specific Ketamine actions. It was concluded that Ketamine inhibits the presynaptic nicotinic receptors responsible for facilitating neurotransmitter release, as well as the direct ligand-gated inward current. Alpha-cobratoxin, a protein with a molecular weight of 7831 and 71 amino acids, and its homologue, alpha-bungarotoxin (BTX), preferentially target the alpha7 and alpha1 nicotinic acetylcholine receptors (NAchR) in nerve and muscle tissue, respectively, and functions by preventing activation of such acetylcholine receptors in pre- and post-synaptic membranes. The toxicity of these molecules is based upon their relative affinity for the receptor which far exceeds that of acetylcholine. Many studies (Miller et al., 1977, Hudson et al., 1983, Lentz et al., 1987, Donnelly-Roberts and Lentz, 1989, Chang et al., 1990, Fiordalisi et al., 1994) have demonstrated various methods for the chemical modification of cobratoxin, by oxidation with substances such as hydrogen peroxide, formalin and ozone, which result in an alteration in affinity for the acetylcholine receptor (AChR) and a concomitant loss in toxicity.

The administration of a highly toxic substance such as cobratoxin for therapeutic purposes is fraught with obvious difficulties, even when highly diluted. As a diluted substance, its potential effectiveness is reduced. As taught by Sanders, removal of the toxicity of cobratoxin can be achieved by exposure to heat, formalin, hydrogen peroxide, performic acid, ozone or other oxidizing/reducing agents. The result of exposure of cobratoxin to these agents is the modification of amino acids as well as the possible lysis of one or more disulfide bonds. Tu (1973) has demonstrated that the curaremimetic alpha neurotoxins of cobra and krait venoms lose their toxicity upon either oxidation or reduction and alkylation of the disulfide bonds which has been confirmed by Hudson et al (1983). Loss of toxicity can be determined by the intraperitoneal injection of excess levels of the modified cobratoxin into mice; in general a 1 mL volume containing 0.5-1 mg of modified cobratoxin is tested, which represents a minimum of a 400-fold reduction of toxicity. Alternatively, loss of toxicity can be evaluated by depression of binding of the modified neurotoxin to acetylcholine receptors (AChR) in vitro.

Another aspect of the present invention relates to the production of drug product as an improvement over the prior art of Sanders as earlier described hereinabove. It uses a modification of the known Bouquet technique (Ann. Inst. Pasteur 66:379-396, 1941). According to his procedure, a solution of the venom in a suitable solvent, especially water, was prepared. While the particular concentration of venom in the solution was not critical, up to about 3% by weight solution was feasible. An antifoam was added to the solution, since snake venoms caused solutions to foam. Any nontoxic inert antifoam was proposed. CP hyperoxide (30% solution) was added, along with a catalyst for the activation of the hydrogen peroxide, such as copper sulfate. Since detoxification was suggested to proceed on the basic side, the PI was adjusted to above 7, but preferably less than 10, with a suitable base such as a metal or alkaline earth hydroxide, carbonate or the like, e.g., sodium hydroxide.

The temperatures of the reaction was carried out at 37° C. though temperatures outside of this range were permissible, but lower temperatures prolonged the period required for detoxification and higher temperatures were believed to cause unacceptable amounts of denaturing of the venom. Occasionally the mixture was stirred. Following about 30 days, especially 6 and 16 days under the foregoing conditions, depending upon the temperature and the particular venom, detoxification was deemed accomplished and the venom was modified for purposes of Sanders invention.

The detoxification reaction was then stopped by adding an enzyme to remove the remaining hydrogen peroxide. Catalase (CP) was the most convenient for this purpose. Since the modified venom produced by Sanders contained ions (e.g., copper sulfate) added as part of the detoxification procedure, and which were considered undesirable, it was preferred that these ions were removed from the modified venom product. The ions were removed by dialysis with semi-permeable membranes. The detoxified solution was simply contained in a semi-permeable membrane, such as cellulose acetate, and the membrane with its contents were submerged in a tank of phosphate buffer—sodium chloride solution, pH 6.8, to cause transfer of the undesired ions from the modified venom solutions of the salt bath. Suitably, this was carried out at room temperature for approximately one day. The modified venom was preferably filtered, e.g. through a series of graded pore diameter membranes, particularly a series including a final membrane with a very small average pore diameter, e.g., about 0.22 microns, to insure sterility. Also prior to final filtration, it was preferred to adjust the pH of the bulk product to less than 7, e.g., 6.8, by the use of food grade nontoxic acids, such as mineral acids, acetic acid, lactic acid and the like. The particular pH was preferred to be at a pH above 4 and below 7.

The conversion of neurotoxins with hydrogen peroxide is relatively simple and can be achieved at relatively high protein concentrations (10 mg/ml). The reactive species is cheap and abundant. The process employed by Sanders above required the addition of some agents which preferably required removal post reaction. Agents such as catalase, copper sulphate and phosphate buffers. While these agents have proven safe in chronic toxicity tests it is always desirable to reduce the number of chemicals where possible to minimize their effects on the host.

The reaction procedure with hydrogen peroxide occurs over the course of 3-4 days but loss of toxicity may be achieved within 24 hours. Miller's studies (1977) have shown that with continued oxidation, the loss of the tryptophan residue can be observed. This coincides with the method for following the reaction of neurotoxins with ozone (Chang et al, 1990; Mundschenk patent number U.S. Pat. No. 5,989,857). Studies conducted by Miller suggest that the loss of toxicity is due mainly to the reduction in the number of disulphide bonds.

It was believed that the reaction of hydrogen peroxide with the neurotoxin could be achieved in the absence of copper sulphate, phosphate buffers and catalase. It is interesting that copper sulphate is reported to inhibit the activity catalase (communication from Sigma-Aldrich). The presence of copper sulphate was found to be essential for the reaction and that the concentration of this compound present in the reaction mixture did not inhibit catalase. The reaction can proceed in a solution of saline (or phosphate buffered saline, PBS) for injection/irrigation with hydrogen peroxide and protein being present. The major concern is that residual hydrogen peroxide is removed which may be achieved with heat, by raising the solution temperature to 70° C. A platinum stir bar may also suffice as a method to remove hydrogen peroxide. Heat, in excess of 70° C., may not be appropriate in situations where the protein concentration is greater than 1 mg/ml. The reaction can be allowed to continue for 72 hours at which time the solution can be heated to 60° C. to drive of the peroxide.

The general formulation is therefore;

| | |
|---|---|
| alpha-neurotoxin or venom | 40 g |
| H₂O₂ (30%) | 80 ml |
| Copper sulphate (1%) | 1 ml |
| Saline (0.9%) or PBS | 3900 ml |
| Total | 4000 ml |

Alpha-neurotoxin solution, i.e. cobratoxin, is filter sterilized to remove bacteria. It can be dissolved in saline and made up to final volume minus H₂O₂ volume. H₂O₂ should be added last while agitating. Final product is 10 mg/ml. Conceivably the protein level can be increased concomitant with an increase in the level of H₂O₂ to yield 20 or 30 mg/ml solutions. There is a 1000 fold molar excess of H₂O₂ relative to neurotoxin. This would increase production while keeping the handling volume to a minimum. The solution needs to be diluted prior to filling and administration usually between 500 and 1000 mcg/ml. Any suitable preservative for parenteral administration can be employed such as methyl paraben, benzalkonium chloride or metacreosol.

For oral administration, a solution of protein mixed with the appropriate ratio of benzalkonium chloride is prepared where the ratio of protein to detergent is 7:1 (w/v). This formulation has an estimated oral efficiency of approximately 40%, requiring more frequent applications than the parenteral format. The preferred formulation uses protein concentrations of 0.025% up to 0.05% with a corresponding benzalkonium chloride concentration of 0.0035% and 0.007% respectively. An aerosol atomizer delivering between 0.07 and 0.1 cc per actuation is preferred to maximize efficiency of delivery and minimize waste. The spray is directed at the back of the throat or applied sub-lingually where the active component is absorbed through the mucosal lining.

In the treatment of pain the administration of modified cobratoxin (or venom) is required regularly, at least once per day extending to several applications. Parenteral (intramuscular or subcutaneous) or oral administration of the modified neurotoxins should deliver at least 10 mcg/day up to a maximum of 3 mg. Studies have shown the average dose to be between 200 and 400 mcg/day for purified neurotoxin preparations and 1-2 mg/day for venom preparations. Higher doses can be employed for more rapid onset of effect with the preferred route being intravenous. For topical applications, the applicable concentration of the present modified neurotoxin range from a minimum of 6 mcg per gram of base up to 1 mg per gram. The applicable concentrations of modified venom are 5 fold greater than that for the purified neurotoxin as the neurotoxin accounts for approximately 15-20% of the composition of the venom. The average drug concentration of 15-30 mcg per gram of base is preferable. The rate of application can range from an infrequent, as needed basis, to several applications per day particularly where the application is for the control of pain. The treatment of oral herpes, shingles and genital herpes may require 4 to 5 topical applications per day in order to reduce pain and speed healing.

### EXAMPLE 1

### Receptor Binding Activity

Natural cobra alpha-neurotoxin is toxic because of its' high affinity binding to acetylcholine receptors (AChR). Oxidation of cobra alpha-neurotoxin abolishes the toxicity of the alpha neurotoxin, as determined by the absence of lethality by IP or IM injection of the modified cobratoxin into mice. Binding of modified cobratoxin into NAchR in vitro has been determined to still occur though with greatly decreased affinity. Modified cobratoxin-AChR binding in vitro is determined by a modification of an enzyme immunoassay (EIA) developed by B. G. Stiles (1991) for the detection of postsynaptic neurotoxins. In the published version of the assay, neurotoxin or oxidized neurotoxin is bound by hydrophobic interaction to the wells of a polystyrene immunoassay plate. After washing of the wells, whole acetylcholine receptor (ACHR) from Torpedo californica isolated by the method of Froehner and Rafto (1979) is placed in the wells and binds to polystyrene bound neurotoxin or oxidized neurotoxin. Bound AChR is then detected by AChR specific antibody. The specificity of binding of ACHR to polystyrene bound modified cobratoxin has been determined by inhibition of binding by carbamylcholine chloride and by native cobratoxin.

Based first upon the natural high affinity binding of un-modified cobratoxin to AChR and also upon our determination of the continued ability of oxidized cobratoxin to bind to AChR, though with greatly reduced affinity, the activity of modified cobratoxin in vivo is assumed to occur at the level of acetylcholine receptors or acetylcholine-like receptors. The binding of modified cobratoxin with eel AChR in vitro forms the basis for the potency assay for these drugs.

Briefly, the derivative modified cobratoxin potency assay is performed as follows. Test and control modified cobratoxin are examined for their ability to block the binding of biotin labeled cobratoxin to torpedo AchR bound to polystyrene in a sequential binding assay. AchR in carbonate buffer is placed in the wells of a 96 well plate and incubated overnight. After washing, the test solution of modified cobratoxin or other nAchR active substance, is added to the wells and incubated at room temperature for three hours, after which the wells are washed. The concentration of the cobratoxin or other substance is determined emperically with respect to labeled cobra toxin, to provide a specific response. Cobratoxin, labeled with biotin is placed in all wells and incubated for one hour and the wells washed. This is followed by a one hour incubation with streptavidin-horseradish peroxidase present in the wells. After washing, ortho-phenylenediamine in citrate buffer is placed in the wells; color development is stopped after 30 minutes by the addition of 4NH2SO4. Absorbance is determined at 490 nm. In this assay, low generated absorbance is indicative of high modified cobratoxin binding, while high generated absorbance is indicative of poor interaction between AchR and modified cobratoxin. Preparations which have exhibited excellent or poor therapeutic capabilities are utilized as controls.

The applicants' experiences in several disorders (Multiple Sclerosis, Amyotrophic Lateral Sclerosis) demonstrate improved function (muscle strength, walking speed) and endurance. The mechanism is assumed to involve mainly presynaptic acetylcholine receptors as outlined in provisional patent application no. 1013-1. Haast (1982) reports that patients receiving neurotoxin combinations reported similar effects. While cobratoxin does bind to the muscle receptor in-vitro very little or no paralysis is observed in mice injected with the toxin which supports the above theory.

### EXAMPLE 2

### Human Subject with Oral Herpes

A 36 year old human male with a history of oral herpes (herpes simplex type 1) assessed the effects of parenterally administered oxidized alpha-cobratoxin on oral lesions. The subject discovered that the injection of the drug reduced pain associated with nasal or labial herpetic lesions when administered at the first indication of a prodrome. Also noted was a reduction in the usual size of the lesion and healing period with continued administration consistent with observations in other clinical studies on herpesvirus.

### EXAMPLE 3

### Human Subject with Metastatic Cancer

A 40 year old human male diagnosed with malignant fibrous histiocytomas originating in his right leg. He underwent standard radiotherapy to control this condition. In the course of the radiation therapy, the patient experienced significant pain in this area. The administration of modified alpha-cobratoxin in an oral format (0.1 cc every 2-3 hours) reduced the pain level to a comfortable level such that the patient could return to work. The oral formulation was employed to control pain for the duration of radiation treatment extending over 11 months.

### EXAMPLE 4

### Human Subject with Metastatic Cancer

A 51 year old human female diagnosed with progressive metastatic melanoma originating on the subjects left shoulder. CT scans revealed extensive dissemination of the cancer throughout the abdomen. Radiation therapy was prescribed which resulted in nausea and pain. Parenteral administration of oxidized alpha-cobratoxin at 100 micrograms every 4-5 hours reduced the side effects of radiation and helped control pain. The patient employed this drug to improve her quality of life for over 12 months when she unfortunately succumbed to the cancer.

### EXAMPLE 5

### Human Subject with ALS

A human male with confirmed ALS was administered autoclaved alpha-cobratoxin in an oral formulation comprising 600 mcg/ml of the neurotoxin and 0.01% Benzalkonium chloride suspended in 0.9% physiological saline. In the absence of anticholinergic therapy the patient reported stiffness and pain upon rising and leg pain during the day. This combined with reduced endurance and strength comprised the symptoms to be followed when assessing the new formulation. Following an overnight abstinence from other anticholinergic drugs, he administered 1 spray sublingually (equivalent to 0.1 ml volume). He noted reduction of pain and increased strength approximately 15 minutes post administration. Administration of the solution throughout the day at 2-3 hour intervals provided satisfactory improvements in strength, endurance and relief from pain equivalent to prior therapeutic modalities.

### EXAMPLE 6

### Human Subject with MS

A human female with confirmed MS was administered oxidized alpha-cobratoxin in a parenteral formulation comprising 500 mcg/ml of the neurotoxin and 0.001% Benzalkonium chloride suspended in 0.9% physiological saline. This patient experienced similar symptoms described for Example 6. Following an overnight abstinence from other anticholinergic drugs, she administered 1 injection (equivalent to 0.5 ml volume). She noted improved pain and strength approximately 20 minutes post administration. Administration of the solution throughout the day at intervals of 4-5 hours provided satisfactory improvements in strength, endurance and relief from pain.

### EXAMPLE 7

### Human Subject with RA

A human female, aged 93 with diagnosed RA which produced pain in neck, hands, wrists and knees utilized oxidized alpha cobratoxin in a cream base at a concentration of 15-60 ug modified cobratoxin per gram of cream base. Application was on an as needed basis. The patient observed a decrease in pain characterized as allowing her to feel comfortable. Along with the loss of pain was an increase in mobility in the areas to which the therapeutic was applied. The therapeutic produced a positive effective within 20 minutes and relief lasted several hours.

As noted in the summary of the invention above, there are provided alternative methods of drug production. These include heat treatment of cobra toxin and venoms. These novel methods of production give the option of generating proteins with subtle differences that have great importance to their application. Excessive exposure to heat is a mechanism that can be employed to investigate stability and heat-stress studies are commonly employed to assess the heat sensitivity of a protein and to simulate the passage of time. Cobratoxin (CTX) can be autoclaved (121° C., 20 minutes) in water or saline at concentrations of up to 900 mcg/ml though the initial experiments were conducted with solutions of 100 mcg/ml. Following this exposure the container and solution remain intact and clear though with some precipitation. At lower concentrations very little precipitation was observed and there were no obvious indications of deterioration. When measured, the protein concentration did not change significantly even when the level of precipitation appeared excessive. When examined by PAGE the autoclaved CTX migrated similarly to that being in a reduced state. The intensity of the staining was reduced though the same quantity of protein was loaded for each pair suggesting an event like oxidation was responsible for the effects observed. There was no discernible difference in the resulting product when autoclaving was conducted in distilled water or saline for injection. The presence of a preservative did not appear to alter the appearance of the autoclaved protein when analyzed by PAGE. Also, when stored over time (2 years) in solution, CTX appears to undergo a transformation to a more unfolded form. These changes were correlated with reduced toxicity in mice. It is interesting to note that the native CTX treated under these conditions did not appear to degrade to smaller (<7900 d) peptide fragments. CTX was convenient to employ for these studies because potency and toxicity are interwoven. It was not determined at the time whether autoclaved CTX demonstrated any anti-viral activity. This study suggests that CTX maintains an overall molecular weight of circa 8,000 d following autoclaving though some smaller fragments can be observed below 8,000 d. Additionally UV analyses of the autoclaved samples indicate there are no observable changes in the absorption characteristics, the tryptophan residue remaining intact which suggests that this was a milder form of oxidation that hydrogen peroxide (Miller et al, 1977) or ozone (Chang et al., 1990). The neurotoxin's resistance to heat permits the use of heat as a modification to the original formula developed by Sanders. The instability of hydrogen peroxide to heat also permits the utilization of heat as a method to drive off excess hydrogen peroxide when the reaction with venom or purified neurotoxins is deemed complete. However unless gentle heat is employed or the solution is diluted the use of high temperature should be avoided. Lower temperature elevations are advised in solutions containing proteins concentrations greater than 1 mg/ml.

The injection of autoclaved cobratoxin (600 mcg/ml, 0.01% BC) into 4 mice (sc, 50 mcl-30 mcg) produced no toxic indications and no deaths over 3 days of observations. Injection of the control, non-autoclaved cobratoxin (600 mcg/ml, 0.01% BC) into 4 mice (sc, 50 mcl-30 mcg) resulted in deaths averaging 20.5 minutes. The injection of solution autoclaved at 300 and 900 mcg/ml also failed to kill mice.

While the invention has been described, and disclosed in various terms or certain embodiments or modifications which it has assumed in practice, the scope of the invention is not intended to be, nor should it be deemed to be, limited thereby and such other modifications or embodiments as may be suggested by the teachings herein are particularly reserved especially as they fall within the breadth and scope of the appended claims.

1. A method of treatment of a subject of one of animals and humans experiencing pain comprising administering to the subject a mitigating amount of a detoxified and neurotropically active modified venom neurotoxin composition using one of parenterally, topically and orally.
